(19) United States
(12) Patent Application Publication
(10) Pub. No.: US 2012/0315246 A1
(43) Pub. Date: Dec. 13, 2012

(54) Compositions and methods for modulating inflammatory and/or immune responses

(76) Inventors: Randall G. Rupp, Swanton, VT (US); Richard A. Banas, Turtle Creek, PA (US)

(21) Appl. No.: 13/485,972

(22) Filed: Jun. 1, 2012

Related U.S. Application Data
(60) Provisional application No. 61/520,660, filed on Jun. 13, 2011.

(51) Int. Cl. A61K 35/54; A61P 29/00; A61K 38/21; A61P 37/06; A61K 39/00; A61K 39/395

(52) U.S. Cl. 424/85.4; 424/582; 424/184.1; 424/130.1; 424/141.1; 424/143.1; 424/144.1

(57) ABSTRACT

The invention is directed to novel compositions and methods for modulating inflammatory and/or immune responses. Such novel compositions are derived from extraembryonic cells (herein referred to as EE cells) including but not limited to extraembryonic HLA-G positive cells (herein referred to as extraembryonic HLA-G positive or “EHP” cells) and Amnion-derived Multipotent Progenitor cells (herein referred to as AMP cells), alone or in combination with each other and/or in combination with various matrices and/or devices and/or other suitable active agents. The novel methods of modulating inflammatory and/or immune responses utilize such novel compositions.

### CROSS-REFERENCE TO RELATED APPLICATIONS

This application claims priority under 35 USC § 119(e) of U.S. Provisional Application No. 61/520,660, filed Jun. 13, 2011, the entirety of which is incorporated herein by reference.

### FIELD OF THE INVENTION

The field of the invention is directed to novel compositions and methods for modulating inflammatory and/or immune responses. Such novel compositions are derived from extraembryonic cells (herein referred to as EE cells) including but not limited to extraembryonic HLA-G positive cells (herein referred to as extraembryonic HLA-G positive or “EHP” cells) and Amnion-derived Multipotent Progenitor cells (herein referred to as AMP cells), alone or in combination with each other and/or in combination with various matrices and/or devices and/or other suitable active agents. The novel methods of modulating inflammatory and/or immune responses utilize such novel compositions.

### DESCRIPTION OF RELATED ART

U.S. Published Application No. 2006-026337 (incorporated herein by reference) discloses the immunomodulatory properties of multipotent adult progenitor cells, called MAPCs, and uses thereof

Ueta, M., et al., (Clin Exp Immunol 2002; 129:464-470) describe the immunosuppressive properties of decellularized amniotic membrane.

Klyushnenkova, E., et al., (Journal of Biomedical Science, 2005, 12:47-57) describe T cell responses to allogeneic human mesenchymal stem cells, called MSCs.

Williams, M. (Journal of Hematotherapy & Stem Cell Research, 2003, 12:757-758) discusses the functional expression of HLA-G and whether it can be exploited for successful stem cell transplantation and engraftment.

Gotherstrom, C., et al., (The Hematology Journal, 2005, 90(8):1017-1026) disclose that adult bone marrow-derived mesenchymal stem cells do not express HLA-G protein.

PCT/US2008/00396 describes extraembryonic cells and Amnion-derived Multipotent Progenitor (AMP) cells, and/or cell lysates and/or conditioned media derived therefrom, that are useful agents capable of treating HVG, GVHD, as well as other immune and/or inflammatory diseases and disorders.

Banas, R. A., et al, (Human Immunology (2008) 69, 321-328) describe the immunogenicity and immunomodulatory effects of Amnion-derived Multipotent Progenitor (AMP) cells.

### BACKGROUND OF THE INVENTION

An important consideration in stem cell and organ transplant therapies is graft tolerance. In humans, the protein expression of the cell surface marker HLA-G was originally thought to be restricted to immune-privileged sites such as placenta, as well as related cells, including some isolated from amniotic fluid, placental macrophages, and cord blood, thus implicating its role in maternal-fetal tolerance (Urosevic, M. and Dummer, R. (2002) ASHI Quarterly; 3rd Quarter 2002:106-109). Additionally, studies involving heart-graft acceptance have suggested that the protein expression of HLA-G may enhance graft tolerance (Lila, N., et al. (2000) Lancet 355:2138; Lila, N. et al. (2002) Circulation 105:1949-1954). HLA-G protein is not expressed on the surface of undifferentiated or differentiated embryonic stem cells (Drukker, M, et al. (2002) PNAS 99(15):9864-9869). Thus, it is generally considered desirable that stems cells intended for cell-based therapies express HLA-G protein.

The transfer of living cells, tissues, or organs from a donor to a recipient, with the intention of maintaining the functional integrity of the transplanted material in the recipient defines transplantation. A major goal in solid organ transplantation is the permanent engraftment of the donor organ without a graft rejection immune response generated by the recipient, while preserving the immunocompetence of the recipient to respond to other foreign antigens. Typically, in order to prevent host rejection responses, nonspecific immunosuppressive agents such as cyclosporine, methotrexate, steroids and FK506 are used. These agents must be administered on a daily basis and if stopped, graft rejection usually results. Despite the use of immunosuppressive agents, chronic graft rejection still remains a major source of morbidity and mortality in human organ transplantation.

Among the most prominent adverse reactions encountered as a result of transplant therapies are (i) the host versus graft response (“HVG”) (rejection of the transplant by an immune competent host), and (ii) graft versus host disease (“GVHD”) (which occurs primarily in an immunocompromised host when it is recognized as non-self by immunocompetent cells in the graft). Graft rejection in a host can be avoided by perfectly matching the donor and the host tissue. However, perfect matches are virtually non-existent (with the exception of identical twins). One potential way around this is the use of autologous (syngeneic) tissue. Unfortunately, the host tissue is often not suitable or was not collected prior to need. In fact, the need for the transplant therapy is frequently to replace damaged tissue in the host. This means that the use of autologus (syngeneic) tissue is not generally useful in practical applications.

Another option is matching an allogeneic donor and host as closely as possible using blood and/or tissue typing. Unfortunately, even the closest of matches does not prevent serious HVG, so allogeneic transplant therapies require immunosuppression with immunosuppressive drugs.

Another approach to avoid HVG and its complications in transplant therapies is to disable the immune system of the recipient host. A draw back to such immunoablation or suppression is that it compromises the host's immune defenses such that the host is readily susceptible to infections, a major cause of morbidity and mortality among transplant patients. Compromising the host immune system also causes or exacerbates GVHD. GVHD occurs when donor tissue contains immunocompetent cells that recognize MHC proteins of the host as non-self. This activates T-cells which in turn secrete inflammatory cytokines, such as IL-2, interferon gamma, and TNF alpha, which trigger an immune attack on host targets including the skin, GI tract, liver, and lymphoid organs (Ferrara and Deeg, 1991). GVHD is particularly a problem in bone marrow transplants, where it has been shown to be mediated primarily by T lymphocytes (Grebe and Streilein, 1976).

A number of immunosuppressive drugs have been developed and are in use to prevent and/or treat these immune system dysfunctions. Unfortunately, none of the immunosuppressive drugs currently available are entirely effective and all of them have serious drawbacks and deleterious side effects. Glucocorticoids, which are used primarily to treat inflammation and inflammatory diseases, are known to be immunosuppressive and are considered be the best primary treatment for HVG and GVHD. They inhibit T-cell proliferation and T-cell-dependent immune responses. Drugs that act on immunophilins (i.e. cyclosporine, tacrolimus, sirolimus) can be effective in reducing adverse immune reactions in transplant patients, but they also weaken the immune system so much that patients are left highly vulnerable to infections. Cytostatics (i.e. methotrexate, azathiopine, mercatopurine, and cytotoxic antibiotics) are also widely used either alone or in combination with other drugs. They cause a variety of side effects, some of which can be deleterious to the patient. Antibodies (polyclonals and monoclonals such as anti-T-cell receptor (CD23) and anti-IL2 receptor (CD25) antibodies) have also been used. Many other drugs have also been used (i.e. interferon, opioids, TNF binding proteins, mycophenolate, and small biological agents such as FTY720). None of the immunosuppressive drugs, whether used alone or in combination with other agents, are fully effective and all of them generally leave patients still susceptible to HVG and GVHD and weaken their ability to defend against infection. Furthermore, all of these drugs cause serious side effects including gastrointestinal toxicity, nephrotoxicity, hypertension, myelosuppression, and hepatotoxicity, to name a few.

Clearly, a more specific type of immune suppression without the drawbacks listed above would be ideal. For example, an agent that can suppress or eliminate alloreactive T-cells, specifically, would be effective against HVG and GVHD (at least for allogeneic grafts) without the negative side effects that occur with agents that generally attack and compromise the immune system. However, to date, no such agent(s) have been developed. Therefore, it is an object of the present invention to fulfill this unmet need.

### BRIEF SUMMARY OF THE INVENTION

In accordance with the present invention, Applicants have discovered that plasma membranes derived from extraembryonic cells (EE cells) including but not limited to extraembryonic HLA-G positive cells (EHP cells) and Amnion-derived Multipotent Progenitor cells (AMP cells), alone or in combination with each other and/or other suitable active agents, are useful agents capable of treating HVG, GVHD, as well as many other immune and/or inflammatory diseases and disorders. The membrane compositions of the present invention are positive for HLA-G and are negative for MHC Class II antigens. Furthermore, because the compositions are plasma membranes derived from cells rather than living cells themselves, concerns associated with cell transplant such as teratoma formation are greatly reduced or eliminated. In addition, the compositions of the invention are easier to make in large quantities, easier to store, and easier to distribute compared to living cells which require special handling, thus greatly reducing manufacturing costs.

Accordingly, a first aspect of the invention is a composition comprising substantially purified extraembryonic (EE) cell plasma membranes. In a specific embodiment, the EE cell plasma membranes are selected from the group consisting of extraembryonic HLA-G positive (EHP) cell plasma membranes and Amnion-derived Multipotent Progenitor (AMP) cell plasma membranes. In another embodiment, the EE, EHP or AMP cell plasma membrane compositions are pharmaceutical compositions. Still another embodiment is a kit comprising the pharmaceutical compositions.

A second aspect of the invention is a method of making a composition comprising substantially purified EE cell plasma membranes comprising isolating EE cells from extraembryonic tissue; treating the EE cells such that their plasma membranes are separated from the other components of the EE cells; and recovering the separated plasma membranes. In a specific embodiment of this aspect of the invention, the EE cells are selected from the group consisting of EHP cells and AMP cells.

A third aspect of the invention is a method of down-regulating an immune response or an inflammatory response in an subject in need thereof, such method comprising administering to the subject an effective amount of a composition selected from the group consisting of EE cell plasma membranes, EHP cell plasma membranes and AMP cell plasma membranes. In a specific embodiment, the immune response is an autoimmune response which is selected from the group consisting of Type I diabetes, multiple sclerosis, systemic lupus erythematosus, Grave's disease, autoimmune hemolytic anemia, bullous pemphigoid, Hashimoto's thyroiditis, myasthenia gravis, pemphigus, and pernicious anemia. In another specific embodiment the immune response is an allogeneic immune response which is selected from the group consisting of graft versus host disease and host versus graft disease. In yet another specific embodiment the inflammatory response is selected from the group consisting of an inflammatory disease of the integument, an inflammatory bowel disease and a rheumatic disease.

A fourth aspect of the invention is one in which the compositions of aspect one are co-administered with one or more active agents, wherein the active agent is selected from the group consisting of a corticosteroid, a cyclosporine, a tacrolimus, a sirolimus, a methotrexate, a azathiopine, a mercatopurine, a cytotoxic antibiotic, a polyclonal antibody, a monoclonal antibody, an interferon, an opioid, a TNF binding protein, a mycophenolate, and FTY720. In a particular embodiment, the monoclonal antibody is selected from the group consisting of an anti-T-cell receptor (CD23) and an anti-IL2 receptor (CD25) antibody.

Other features and advantages of the invention will be apparent from the accompanying description and the claims. The contents of all references, pending patent applications and published patents, cited throughout this application are hereby expressly incorporated by reference. In case of conflict, the present specification, including definitions, will control.

### DEFINITIONS

As defined herein “isolated” refers to material removed from its original environment and is thus altered “by the hand of man” from its natural state.

As used herein, the term “protein marker” means any protein molecule characteristic of a cell or cell population. The protein marker may be located on the plasma membrane of a cell or in some cases may be a secreted protein.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cell types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cells” or “EE cells” means a population of cells derived from the extraembryonic tissue.

As used herein, the term “EHP cells” means a population of cells derived from the extraembryonic tissue which have the characteristics of being HLA-G positive upon isolation, are MHC Class II negative, do not express the co-stimulatory molecules CD80 and CD86 and are not MAPCs as described in US Published Patent Application No. 20060263337.

As used herein, the term “Amnion-derived Multipotent Progenitor cell” or “AMP cell” means a population of epithelial cells that are derived from the epithelial layer of the amnion. In addition to the characteristics described above for EHP cells, AMP cells have the following characteristics. They have not been cultured in the presence of any non-human animal materials, making them and cell products derived from them suitable for human clinical use as they are not xeno-contaminated. AMP cells are cultured in basal medium supplemented with human serum albumin. In a preferred embodiment, the AMP cells secrete the cytokines VEGF, Angiogenin, PDGF and TGFβ32 and the MMP inhibitors TIMP-1 and TIMP-2. The physiological range of the cytokine or cytokines in the unique combination is as follows: ˜5-16 ng/mL for VEGF, ˜3.5-4.5 ng/mL for Angiogenin, ˜100-165 μg/mL for PDGF, ˜2.5-2.7 ng/mL for TGFβ32, ˜0.68 μg mL for TIMP-1 and ˜1.04 μg/mL for TIMP-2. AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the AMP cells react with antibodies to low molecular weight cytokeratins, confirming their epithelial nature. Freshly isolated amnion-derived cells, from which AMP cells are isolated, will not react with antibodies to the stem/progenitor cell markers c-kit (CD117) and Thy-1 (CD90). Even upon culture, AMP cells remain negative for c-kit (CD117). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells.

The term “composition of extraembryonic cells” as used herein includes the cells and compositions described in this application and in U.S. 2003/0235563, U.S. 2004/0161419, U.S. 2005/0124003, U.S. Provisional Application Nos. 60/666,949, 60/699,257, 60/742,067, 60/813,759, U.S. application Ser. No. 11/333,849, U.S. application Ser. No. 11/392,892, PCT U.S. 06/011392, U.S. 2006/0078993, PCT/U.S. 00/40052, U.S. Pat. No. 7,045,148, U.S. 2004/0048372, and U.S. 2003/0032179, the contents of which are incorporated herein by reference in their entirety.

As used herein, the term “plasma membrane” means the phospholipid bilayer structure that comprises the external boundary of a cell. Plasma membranes have an external or extracellular face and an internal or intracellular face. Each face has unique features and components (i.e. proteins) associated with it that give it its unique properties. In addition, adherent cells exhibit polarity, meaning the aspect of the cell that is attached to the basement membrane, called the basal face, has different features than the aspect of the cell that is adluminal, called the apical face.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “expanded”, in reference to EHP cell compositions, means that the EHP cell population constitutes a significantly higher concentration of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of cells. As used herein “passage” or “passaging” refers to subculturing of cells. For example, cells isolated from the amnion are referred to as primary cells. Such cells are expanded in culture by being grown in the growth medium described herein. When such primary cells are subcultured, each round of subculturing is referred to as a passage.

As used herein, “specific activity” means the specific activity of EHP cell plasma membranes, including AMP cell plasma membranes, and is determined by calculating a 50% inhibition dosage (ID₅₀). For example, using a standard allogeneic-antigen MLR, the 100% response is calculated by determining the PBMC responder response to the mismatched stimulator without addition of AMP cell plasma membranes. Then, AMP cell plasma membranes are titered into the MLR at 1:2 serial dilutions. The amount of AMP cell plasma membrane required to half the 100% response is reported as the ID₅₀.

As used herein, the term “substrate” means a defined coating on a surface that cells attach to, grown on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

The term “transplantation” refers to the administration of a cell composition either in an undifferentiated, partially differentiated, or fully differentiated form into a human or other animal.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “allogeneic” means variation in alleles among members of the same species. As used herein, the term “syngeneic” means genetically identical members of the same species.

As used herein, the terms “immunosuppressive drugs” or “immunosuppressants” are agents that are used in immunosuppressive therapy to inhibit, prevent or modulate activity of the immune system.

As used herein, the term “GVHD” refers to graft versus host disease, which means the processes that occur primarily in an immunocompromised host when it is recognized as non-self by immunocompetent cells of a graft.

As used herein, the term “HVG” refers to host versus graft response, which means the processes which occur when a host rejects a graft. Typically, HVG is triggered when a graft is recognized as foreign (non-self) by immunocompetent cells of the host.

As used herein, the terms “inflammation” or “inflammatory response” means the reaction that occurs in affected cells and adjacent tissues in response to an injury or abnormal stimulation caused by a physical event or a chemical or biologic substance.

As used herein, the term “immune response” means the cells, tissues and protein factors (i.e. cytokines) involved in recognizing and attacking foreign substances within the body of an animal.

As used herein, the term “pharmaceutically acceptable” means that the components, in addition to the therapeutic agent, comprising the formulation, are suitable for administration to the patient being treated in accordance with the present invention.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

As used herein “subject” may mean either a human or non-human animal.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

### DETAILED DESCRIPTION

In accordance with the present invention there may be employed conventional molecular biology, microbiology, and recombinant DNA techniques within the skill of the art. Such techniques are explained fully in the literature. See, e.g., Green and Sambrook, 2012, “Molecular Cloning: A Laboratory Manual, 4^{th} Edition”, Cold Spring Harbor University Press.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### Therapeutic Applications

Applicants have previously demonstrated that relatively small amounts of EHP cells, including AMP cells, can down-regulate inflammatory and immune responses (see PCT/U.S. 2008/00396 and Banas, R. A., et al, (Human Immunology (2008) 69, 321-328) each incorporated herein by reference). Now, Applicants describe herein, for the first time, the use of plasma membranes derived from EE cells, including plasma membranes derived from EHP cells, and in particular, plasma membranes derived from AMP cells, to down-regulate inflammatory and immune responses. Accordingly, various aspects and embodiments of the invention provide novel compositions and methods for down-regulating, adverse and/or abnormal inflammatory and immune responses, such as those that occur in transplantation therapies. The low immunogenicity of allogeneic EHP cell plasma membranes, including AMP cell plasma membranes, their ability to down-regulate adverse and/or abnormal inflammatory and/or immune responses, and their high specific activity (as defined in the definitions and elsewhere herein) makes them particularly valuable for adjunctive therapies in the treatment of conditions in which adverse and/or abnormal inflammatory and/or immune responses are present. EHP cell plasma membranes, including AMP cell plasma membranes, are particularly useful as immunosuppressive adjunctive therapeutics for treating adverse immune responses that occur in transplantation therapy (i.e. HVG and GVHD). Other immune disorders and diseases are discussed in more detail elsewhere herein. EHP cell plasma membranes, including AMP cell plasma membranes, further can be useful in adjunctive immunosuppressive therapy in the treatment of certain inflammatory conditions. Such conditions are discussed in greater detail below and elsewhere in the specification.

Using the methods described herein for the isolation, preparation and characterization of EHP cell plasma membranes, including AMP cell plasma membranes, together with the disclosure herein on the immune-modulating properties of EHP cells, including AMP cells, the novel plasma membrane compositions can be used to down-regulate, prevent, suppress, or diminish immune disorders, dysfunctions, or diseases, including, for example, adverse immune reactions, such as those that result from other therapies, including those that complicate transplantation therapies, such as HVG and GVHD. Such disorders, dysfunctions, and diseases also include congenital immune disorders and autoimmune diseases, among others.

EHP cell plasma membranes, including AMP cell plasma membranes, are useful as both primary and adjunctive therapeutic agents. EHP cell plasma membranes, including AMP cell plasma membranes, can be used therapeutically alone or in combination with each other and/or together with other agents; can be administered before, during, and/or after such other agents; can be administered before, during, and/or after a transplant. If administered during transplant, EHP cell plasma membranes, including AMP cell plasma membranes, can be administered together with the transplant material, or separately. If separately administered, the EHP cell plasma membranes, including AMP cell plasma membranes, can be administered sequentially or simultaneously with the transplant material. Furthermore, EHP cell plasma membranes, including AMP cell plasma membranes, may be administered in advance of the transplant material and/or after the transplant material. Other agents that can be used in conjunction with EHP cell plasma membranes, including AMP cell plasma membranes, in transplantation therapies in particular, include immunomodulatory agents. A variety of such agents are described elsewhere herein. In certain embodiments of the invention, the immunomodulatory agents are immunosuppressive agents, such as those described elsewhere herein.

In certain embodiments the EHP cell plasma membranes, including AMP cell plasma membranes, are administered by injection, such as by intravenous injection; are encapsulated for administration; are administered in situ (i.e. solid organ transplantation and organ repair). These and other forms of administration are discussed in detail elsewhere herein. In some embodiments of the invention, EHP cell plasma membranes, including AMP cell plasma membranes, are administered in doses measured by the ratio of EHP cell plasma membrane, including AMP cell plasma membrane (weight) to body mass (weight). Alternatively, they can be administered in doses of a fixed number. Dosing, routes of administration, formulations, and the like are discussed in greater detail elsewhere herein.

Because of their novel immunomodulatory properties, EHP cell plasma membranes, including AMP cell plasma membranes, each alone or in combination, and/or in combination with other active agents, are particularly useful in the prevention and/or the amelioration of inflammatory responses. Inflammatory responses are characterized by dilation of blood vessels in the affected area resulting in increased blood flow to the area. If at the surface, it makes skin look red and feel warm. Capillaries in the area become more permeable allowing fluid to seep into the surrounding tissue resulting in edematous swelling around the infected site. The swelling and the effects of some of the chemicals released, including inflammatory cytokines, results in pain. Hence clinical characteristics of the inflammatory response are known as redness, heat, edema and pain. The inflammatory response occurs as a result of chemical messages produced by “mediators”. There are two major classes of mediators: 1) cell-derived mediators which are produced by leukocytes (white blood cells) and 2) plasma-derived mediators which are found in the blood plasma. Cell-derived mediators include arachidonic acid derivatives (i.e. prostaglandins and leukotrines), cytokines, lymphokines and monokines, interleukin, platelet activating factor (PAF), histamine and bradykinin. Plasma-derived mediators include complement and interferons. Inflammatory responses may be genetic in nature (i.e. CAPS, see below for details). They may be environmentally induced or virally induced. Non-limiting examples of diseases having inflammatory responses which are suitable for treatment with EHP cell plasma membranes, including AMP cell plasma membranes, each alone or in combination, including in combination with other active agents, include the following:

Inflammatory Diseases of Integument which include psoriasis and eczema (i.e. atopic dermatitis). Psoriasis is an inflammatory skin condition. There are five types, each with unique signs and symptoms. Between 10% and 30% of people who develop psoriasis get a related form of arthritis called “psoriatic arthritis,” which causes inflammation of the joints. Plaque psoriasis is the most common type of psoriasis. About 80% of people who develop psoriasis have plaque psoriasis, which appears as patches of raised, reddish skin covered by a silvery-white scale. These patches, or plaques, frequently form on the elbows, knees, lower back, and scalp. However, they can occur anywhere on the body. The other types are guttate psoriasis (small, red spots on the skin), pustular psoriasis (white pustules surrounded by red skin), inverse psoriasis (smooth, red lesions form in skin folds), and erythrodermic psoriasis (widespread redness, severe itching, and pain). Regardless of type, psoriasis usually causes discomfort. The skin often itches, and it may crack and bleed. In severe cases, the itching and discomfort may keep a person awake at night, and the pain can make everyday tasks difficult. Psoriasis is a chronic, meaning lifelong, condition because there is currently no cure. People often experience flares and remissions throughout their life. Controlling the signs and symptoms typically requires lifelong therapy. Eczema is a general term encompassing various inflamed skin conditions. One of the most common forms of eczema is atopic dermatitis. Approximately 10-20% of the world population is affected by this chronic, relapsing, and very itchy rash at some point during childhood. Fortunately, many children with eczema find that the disease clears and often disappears with age. In general, atopic dermatitis will come and go, often based on external factors. Although its cause is unknown, the condition appears to be an abnormal response of the body's immune system. In people with eczema, the inflammatory response to irritating substances overacts, causing itching and scratching. Eczema is not contagious and, like many diseases, currently cannot be cured. However, for most patients the condition may be managed well with treatment and avoidance of triggers.

Inflammatory Bowel Diseases (IBDs) are chronic inflammatory diseases of the GI tract of unknown etiology, and include ulcerative colitis (UC) and Crohn's disease (CD), which is also referred to as regional enteritis, terminal ileitis, or granulomatous ileocolitis. Ulcerative colitis is an inflammatory disease of the large intestine, also called the colon. In ulcerative colitis, the inner lining (mucosa) of the intestine becomes inflamed and develops ulcers (an ulcer is a sore, which means it's an open, painful wound). Ulcerative colitis is often the most severe in the rectal area, which can cause frequent diarrhea. Mucus and blood often appear in the stool if the lining of the colon is damaged. Crohn's disease differs from ulcerative colitis in the areas of the bowel it involves—it most commonly affects the last part of the small intestine called the terminal ileum and parts of the large intestine. However, Crohn's disease isn't limited to these areas and can attack any part of the digestive tract. Crohn's disease causes inflammation that extends much deeper into the layers of the intestinal wall than ulcerative colitis does. Crohn's disease generally tends to involve the entire bowel wall, whereas ulcerative colitis affects only the lining of the bowel.

### Rheumatic Diseases Encompass Many Diseases Including:

Osteoarthritis—This is the most common type of arthritis, affecting an estimated 21 million adults in the United States. Osteoarthritis primarily affects cartilage, which is the tissue that cushions the ends of bones within the joint. In osteoarthritis, the cartilage begins to fray and may entirely wear away. Osteoarthritis can cause joint pain and stiffness. Disability results most often when the disease affects the spine and the weight-bearing joints (the knees and hips).

Rheumatoid arthritis—This inflammatory disease of the synovium, or lining of the joint, results in pain, stiffness, swelling, joint damage and loss of function of the joints. Inflammation most often affects joints of the hands and feet and tends to be symmetrical (occurring equally on both sides of the body). This symmetry helps distinguish rheumatoid arthritis from other forms of the disease. About 1 percent of the U.S. population (about 2.1 million people) has rheumatoid arthritis.

Juvenile rheumatoid arthritis—This is the most common form of arthritis in childhood, causing pain, stiffness, swelling, and loss of function of the joints. The arthritis may be associated with rashes or fevers, and may affect various parts of the body.

Fibromyalgia—Fibromyalgia is a chronic disorder that causes pain throughout the tissues that support and move the bones and joints. Pain, stiffness, and localized tender points occur in the muscles and tendons, particularly those of the neck, spine, shoulders, and hips. Patients may also experience fatigue and sleep disturbances.

Scleroderma—Also known as systemic sclerosis, scleroderma means literally “hard skin.” The disease affects the skin, blood vessels, and joints. It may also affect internal organs, such as the lungs and kidneys. In scleroderma, there is an abnormal and excessive production of collagen (a fiber-like protein) in the skin or internal organs.

Spondyloarthropathies—This group of rheumatic diseases principally affects the spine. One common form, ankylosing spondylitis, not only affects the spine, but may also affect the hips, shoulders, and knees as the tendons and ligaments around the bones and joints become inflamed, resulting in pain and stiffness Ankylosing spondylitis tends to affect people in late adolescence or early adulthood. Reactive arthritis, sometimes called Reiter's syndrome, is another spondyloarthropathy. It develops after an infection involving the lower urinary tract, bowel, or other organ and is commonly associated with eye problems, skin rashes, and mouth sores.

Gout—This type of arthritis results from deposits of needle-like crystals of uric acid in the joints. The crystals cause inflammation, swelling, and pain in the affected joint, which is often the big toe.

Infectious arthritis—This is a general term used to describe forms of arthritis that are caused by infectious agents, such as bacteria or viruses. Parvovirus arthritis and gonococcal arthritis are examples of infectious arthritis. Arthritis symptoms may also occur in Lyme disease, which is caused by a bacterial infection following the bite of certain ticks. In those cases of arthritis caused by bacteria, early diagnosis and treatment with antibiotics are crucial to get rid of the infection and minimize damage to the joints.

Polymyalgia rheumatica—Because this disease involves tendons, muscles, ligaments, and tissues around the joint, symptoms often include pain, aching, and morning stiffness in the shoulders, hips, neck, and lower back. It is sometimes the first sign of giant cell arteritis, a disease of the arteries characterized by inflammation, weakness, weight loss, and fever.

Polymyositis—This is a rheumatic disease that causes inflammation and weakness in the muscles. The disease may affect the whole body and cause disability.

Psoriatic arthritis—This form of arthritis occurs in some patients with psoriasis, a scaling skin disorder. Psoriatic arthritis often affects the joints at the ends of the fingers and toes and is accompanied by changes in the fingernails and toenails. Back pain may occur if the spine is involved.

Bursitis—This condition involves inflammation of the bursae, small, fluid-filled sacs that help reduce friction between bones and other moving structures in the joints. The inflammation may result from arthritis in the joint or injury or infection of the bursae. Bursitis produces pain and tenderness and may limit the movement of nearby joints.

Tendinitis (Tendonitis)—This condition refers to inflammation of tendons (tough cords of tissue that connect muscle to bone) caused by overuse, injury, or a rheumatic condition. Tendinitis produces pain and tenderness and may restrict movement of nearby joints.

Pelvic inflammatory disease (PID)—Is a general term that refers to infection and inflammation of the upper genital tract in women. It can affect the uterus, fallopian tubes, ovaries, and other organs related to reproduction. The scarring that results on these organs can lead to infertility, tubal (ectopic) pregnancy, chronic pelvic pain, abscesses, and other serious problems. PID is the most common preventable cause of infertility in the United States.

CIAS1-related Autoinflammatory Periodic Syndromes (CAPS)—is a spectrum of rare inherited inflammatory conditions, including Familial Cold Autoinflammatory Syndrome (FCAS), Muckle-Wells Syndrome (MWS), and Neonatal Onset Multisystem Inflammatory Disease (NOMID). These syndromes are characterized by spontaneous systemic inflammation and are termed autoinflammatory disorders. A novel feature of these conditions (particularly FCAS and MWS) is that exposure to mild degrees of cold temperature can provoke a major inflammatory episode that occurs within hours. CAPS are caused by a range of mutations in the gene CIAS1 (also known as NALP3)

Miscellaneous inflammatory diseases include interstitial cystitis, Henoh-Schonlein purpura, and Behcet's syndrome.

Non-limiting examples of diseases having immune responses which are suitable for treatment with EHP cell plasma membranes, including AMP cell plasma membranes, each alone or in combination, including in combination with other active agents, include:

GVHD refers to graft versus host disease, which means the processes that occur primarily in an immunocompromised host when it is recognized as non-self by immunocompetent cells of a graft.

HVG refers to host versus graft response, which means the processes which occur when a host rejects a graft. Typically, HVG is triggered when a graft is recognized as foreign (non-self) by immunocompetent cells of the host.

Juvenile (Type I) Diabetes is caused when the body forms antibodies that attack the beta cells in the islets of Langerhans in the pancreas. Since the beta cells are responsible for producing insulin, Type I diabetics must inject themselves with insulin their entire life.

Systemic lupus erythematosus (also known as lupus or SLE) is an autoimmune disease in which the immune system harms the body's own healthy cells and tissues. This can result in inflammation of and damage to the joints, skin, kidneys, heart, lungs, blood vessels, and brain.

Graves' disease is caused by an abnormal immune system response that attacks the thyroid gland, and causes too much production of thyroid hormones. Risk factors are being a woman over 20 years old, although the disorder may occur at any age and may affect men as well.

Miscellaneous immune disorders include autoimmune hemolytic anemia, bullous pemphigoid, Hashimoto's thyroiditis, myasthenia gravis, pemphigus, and pernicious anemia.

Allergic reactions are sensitivities to a specific substance, called an allergen, which is contacted through the skin, inhaled into the lungs, swallowed, or injected. Allergic reactions vary.

They can be mild or serious. They can be confined to a small area of the body or may affect the entire body. Most occur within seconds or minutes after exposure to the allergen, but some can occur after several hours, particularly if the allergen causes a reaction after it is partially digested. In very rare cases, reactions develop after 24 hours. Anaphylaxis is a sudden and severe allergic reaction that occurs within minutes of exposure. Immediate medical attention is needed for this condition. It can get worse very, very fast and lead to death within 15 minutes if treatment is not received.

One of skill in the art will recognize that HLA-G expression by cells is important in a cell's ability to evade immunosurveillance. Therefore, any cell that expresses HLA-G is potentially useful in practicing the methods of the invention by isolating and preparing that cell's plasma membrane. So, in addition to EHP cell plasma membranes, including AMP cell plasma membranes, suitable cells may include cells such as certain tumor cells (see, for example, Tripathi, P. and Agrawal, S., Cancer Invest 2006, 24(2):178-186; Blaschitz, A., et al., Human Immunology 2000, 61:1074-1085), certain cells in the thymus (see, for example, Mallet, V., et al., Int Immunol 1999, 11:889-898; Mallet, V., et al., Reprod Immunol 1999, 43(2):225-234) and non-progenitor placental-derived HLA-G positive cells (Blaschitz, A., et al., Human Immunology 2000, 61:1074-1085).

### Isolation, Identification and Characterization of EHP Cells Used to Isolate and Prepare EHP Cell Plasma Membranes

Various methods for isolating cells from the extraembryonic tissue, which may then be used to produce the EE cells are described in the art (see, for example, U.S. 2003/0235563, U.S. 2004/0161419, U.S. 2005/0124003, U.S. Provisional Application Nos. 60/666,949, 60/699,257, 60/742,067, 60/813,759, U.S. application Ser. No. 11/333,849, U.S. application Ser. No. 11/392,892, PCT U.S. 06/011392, U.S. 2006/0078993, PCT/U.S. 00/40052, U.S. Pat. No. 7,045,148, U.S. 2004/0048372, and U.S. 2003/0032179).

Identifying EHP cells—Once EE cells are isolated, it is necessary to identify which cells have the characteristics associated with EHP cells. For example, the cells are tested for the presence of HLA-G, the absence of MHC Class II antigens, and the absence of co-stimulatory molecules CD80 and CD86.

### Isolation, Identification and Characterization of AMP Cells

AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human protein (i.e. human serum albumin) and no non-human animal protein; d) selecting AMP cells from the epithelial cell culture, and optionally e) further proliferation of the cells, optionally using additional additives and/or growth factors (i.e. recombinant human EGF). Details are contained in U.S. Publication No. 2006-0222634-A1, which is incorporated herein by reference.

Culturing of the AMP cells—The cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. In specific embodiments, the basal media is IMDM highly enriched basal medium, STEMLINE® T-cell expansion medium or STEMLINE® II hematopoietic stem cell expansion medium, or OPTI-PRO™ serum-free culture medium, or combinations thereof and the human protein is human serum albumin at a concentration of at least 0.5% and up to 10%. In particular embodiments, the human albumin concentration is from about 0.5 to about 2%. In a specific embodiment the human serum albumin is at 0.5%. The human serum albumin may come from a liquid or a dried (powder) form and includes, but is not limited to, recombinant human serum albumin, PLASBUMIN® normal human serum albumin and PLASMANATE® human blood fraction (both made by Talecris Biotherapeutics).

In a most preferred embodiment, the cells are cultured using a system that is free of non-human animal products and substances to avoid xeno-contamination. In this embodiment, the culture medium is IMDM highly enriched basal medium , STEMLINE® T-cell expansion medium or STEMLINE® II hematopoietic stem cell expansion medium, OPTI-PRO™ serum-free culture medium, or DMEM culture medium, with human serum albumin (PLASBUMIN® normal human serum albumin) added up to concentrations of 10%. A particular embodiment is one wherein the human serum albumin is at 0.5%. The invention further contemplates the use of any of the above basal media wherein animal-derived proteins are replaced with recombinant human proteins and animal-derived serum, such as BSA, is replaced with human serum albumin. In preferred embodiments, the media is serum-free in addition to being animal-free.

Optionally, other factors are used. In one embodiment, epidermal growth factor (EGF) at a concentration of between 0-1 μg/mL is used. In a preferred embodiment, the EGF concentration is around 10-20 ng/mL. Alternative growth factors which may be used include, but are not limited to, TGFα or TGFβ2 (5 ng/mL; range 0.1-100 ng/mL), activin A, cholera toxin (preferably at a level of about 0.1 μg/mL; range 0-10 μg/mL), transferrin (5 μg/mL; range 0.1-100 μg/mL), fibroblast growth factors (bFGF 40 ng/mL (range 0-200 ng/mL), aFGF, FGF-4, FGF-8; (all in range 0-200 ng/mL), bone morphogenic proteins (i.e. BMP-4) or other growth factors known to enhance cell proliferation. All supplements are human clinical grade.

In a specific embodiment, the following method is used to obtain selected AMP cells. The cells are plated into plastic tissue culture vessels (i.e. T75 flasks) immediately upon isolation from the amnion. After ˜1-5 days, preferably ˜1-3 days, and most preferably ˜2 days in culture, non-adherent cells are removed from the plastic tissue culture vessel and discarded and the adherent cells are kept. This attachment of cells to a plastic tissue culture vessel is the selection method used to obtain the desired population of AMP cells. Adherent and non-adherent AMP cells appear to have similar cell surface marker expression profiles but the adherent cells have the advantage of possessing greater viability than the non-adherent population of cells and are thus the desired population of AMP cells. Adherent AMP cells are cultured until they reach 13,000-700,000 cells/cm², preferably 53,000-500,000 cells/cm² and most preferably 120,000-300,000 cells/cm². At this point, the cultures are confluent or close to confluent. Suitable cells cultures will reach this number of cells between ˜5-14 days, preferably between 5-9 days. Attaining this criterion is an indicator of the proliferative potential of the AMP cells and cells that do not achieve this criterion are not selected for further analysis and use. Once the AMP cells reach 13,000-700,000 cells/cm², preferably 53,000-500,000 cells/cm² and most preferably ˜120,000-300,000 cells/cm², they are removed from the plastic tissue culture vessel and cryopreserved. This collection time point is called p0.

The AMP cells of the invention are characterized by assaying for secretion of physiologically relevant cytokines and growth factors. Suitable cells are those in which each cytokine or growth factor occurs in the physiological range of ˜5.0-16 ng/mL for VEGF, ˜3.5-4.5 ng/mL for Angiogenin, ˜100-165 μg/mL for PDGF, ˜2.5-2.7 ng/mL for TGFβ32, ·0.68 μg/mL for TIMP-1 and ˜1.04 μg/mL for TIMP-2.

Isolation, preparation and characterization of EHP cell plasma membranes, including AMP cell plasma membranes.

EHP cell plasma membranes, including AMP cell plasma membranes, may be prepared by several methods known in the art (see, for example, Membrane Isolation on Polylysine-coated Beads: Plasma Membrane from HeLa Cells. Carle M. Cohen, Douglas I. Kalish, Bruce S. Jacobson, and Daniel Branton. Journal of Cell Biology, Vol. 75, 1977, 119-134; Plasma Membranes of Mammalian Cells. J. De Pierre, M. Karnovsky. Journal of Cell Biology, Vol. 56, 1973, 275-303; Isolation of the Plasma Membrane from Corneal Endothelial Cells. Z. Suzanne Zam, James Cerda, Frank Polack. Invest. Ophthal. Vis. Sci., Vol. 19, Num. 6, 1980, 648-652; Biomembrane Protocols: Isolation and Analysis. Edited by John Graham and Joan Higgins. Methods in Molecular Biology, Vol 19, 1993 Humana Press, Inc. 59-83.)

Once isolated and prepared, EHP cell plasma membranes, including AMP cell plasma membranes, may be characterized for identification of key membrane immunologic surface markers using flow cytometry (for details, see PCT/U.S. 2008/00396 and Banas, R. A., et al, (Human Immunology (2008) 69, 321-328, each incorporated herein by reference in it's entirety).

In addition, the immunomodulatory mechanisms of EHP cell membranes, including AMP cell membranes, may be evaluated by MLR and co-culture of responding PBMCs to stimulation such as allo-antigen, mitogen, and recall antigen in the presence of different concentrations of EHP cell plasma membranes, including AMP cell plasma membranes (for details, see PCT/U.S. 2008/00396 and Banas, R. A., et al, (Human Immunology (2008) 69, 321-328, each incorporated herein by reference in it's entirety).

### Pharmaceutical Compositions

The present invention provides pharmaceutical compositions of substantially purified EHP cell plasma membranes, including AMP cell plasma membranes, alone or in combination with each other and/or other suitable active agents, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Furthermore, the pharmaceuticals compositions may be combined with one or more active agents. Such other active agents include but are not limited to immunosuppressive agents such as cyclosporine, methotrexate, FK-506, corticosteroids and the like (see below).

### Treatment Kits

The invention also provides for a treatment kit comprising packaging material and a pharmaceutical composition of the invention contained within the packaging material, wherein the pharmaceutical composition comprises a substantially purified EHP cell plasma membranes, including AMP cell plasma membranes, alone or in combination with each other, and optionally in combination with other active agents and wherein the packaging material comprises a label or package insert which indicates that the substantially purified EHP cell plasma membranes, including AMP cell plasma membranes, alone or in combination with each other can be used for treating a variety of disorders including but not limited to HVG, GVHD, and other inflammatory and immune diseases and disorders, allergic reactions, etc.

EHP cell plasma membranes, including AMP cell plasma membranes, alone or in combination with each other, are useful both as primary and adjunctive therapeutic agents and modalities. Such compositions can be used therapeutically alone or together with other agents. Such compositions can be administered before, during, and/or after such agents. Likewise, whether used alone or with other agents, such compositions can be administered before, during, and/or after a transplant or other cell-based therapy. If administered during transplant or other cell-based therapy, the compositions can be administered together with the transplant or other cell-based therapy material, or administered separately. If separately administered, the compositions can be administered sequentially or simultaneously with the transplant or other cell-based therapy. Furthermore, the compositions may be administered in advance of the transplant or other cell-based therapy and/or after the transplant or other cell-based therapy.

In the case of pre-existing conditions, for example autoimmune diseases (i.e. systemic lupus) and inflammatory diseases or disorders (i.e. IBD), the EHP cell plasma membranes, including AMP cell plasma membranes will be administered after the onset of the condition following diagnosis. In these situations, the immune response and/or inflammatory response has already occurred or is continually occurring. The EHP cell plasma membranes, including AMP cell plasma membranes will down-regulate the immune response or inflammatory response such that the condition is controlled and optimally, cured.

EHP cell plasma membranes, including AMP cell plasma membranes, each alone or in combination may be administered with other pharmaceutically active agents. In some embodiments one or more of such agents are formulated together with the EHP cell plasma membrane, including AMP cell plasma membrane compositions for administration. In some other embodiments the compositions and the one or more agents are in separate formulations. In yet some other embodiments the compositions comprising the EHP cell plasma membranes, including AMP cell plasma membranes, each alone or in combination and/or the one or more agents are formulated with regard to adjunctive use with one another.

EHP cell plasma membranes, including AMP cell plasma membranes, each alone or in combination, may be administered in a formulation comprising a immunosuppressive agents, such as any combination of any number of a corticosteroid (i.e. glucocorticoids), cyclosporine, tacrolimus, sirolimus, methotrexate, azathiopine, mercatopurine, cytotoxic antibiotics, polyclonal and monoclonal antibodies such as anti-T-cell receptor (CD23) and anti-IL2 receptor (CD25) antibodies, interferon, opioids, TNF binding proteins, mycophenolate, and small biological agents such as FTY720. Immunosuppressive agents in accordance with the foregoing may be the only such additional agents or may be combined with other agents.

Such agents also include antibiotic agents, antifungal agents, and antiviral agents, to name just a few other pharmacologically active substances and compositions that may be used in accordance with embodiments of the invention. Typical antibiotics or anti-mycotic compounds include, but are not limited to, penicillin, streptomycin, amphotericin, ampicillin, gentamicin, kanamycin, mycophenolic acid, nalidixic acid, neomycin, nystatin, paromomycin, polymyxin, puromycin, rifampicin, spectinomycin, tetracycline, tylosin, zeocin, and cephalosporins, aminoglycosides, and echinocandins.

The EHP cell plasma membranes, including AMP cell plasma membranes, maybe co-administered with various cell types. Examples of other cells types include but are not limited to EHP cells, including AMP cells, embryonic stem cells, adult stem cells, and the like.

Order of administration, formulations, doses, frequency of dosing, and routes of administration of EHP cell plasma membranes, including AMP cell plasma membranes, each alone or in combination, generally will vary with the disorder or disease being treated, its severity, the subject, other therapies that are being administered, the stage of the disorder or disease, and prognostic factors, among others. General regimens that have been established for other treatments provide a framework for determining appropriate dosing of EHP cell plasma membrane, including AMP cell plasma membrane direct or adjunctive therapy. These, together with the additional information provided herein, will enable the skilled artisan to determine appropriate administration procedures in accordance with embodiments of the invention, without undue experimentation.

### Routes and Formulations

Compositions comprising EHP cell plasma membranes, including AMP cell plasma membranes, each alone or in combination, may be formulated in any conventional manner using one or more physiologically acceptable carriers optionally comprising excipients and auxiliaries. Proper formulation is dependent upon the route of administration chosen. The compositions of the invention may be packaged with written instructions for their use in modulating inflammatory and/or immune responses, or restoring other therapeutically important metabolic functions. The compositions may also be administered to the recipient in one or more physiologically acceptable carriers. Carriers for may include but are not limited to solutions of phosphate buffered saline (PBS) or lactated Ringer's solution containing a mixture of salts in physiologic concentrations, or other suitable carriers.

One of skill in the art may readily determine the appropriate concentration of EHP cell plasma membranes, including AMP cell plasma membranes, for a particular purpose. The skilled artisan will recognize that a preferred dose is one which produces a therapeutic effect, such as suppressing and/or otherwise modulating an inflammatory and/or immune response, in a patient in need thereof. Further, proper doses of EHP cell plasma membranes, including AMP cell plasma membranes, and dosing regimens are easily determined by those of skill in the art and need to be empirically determined at time of use based on several variables including but not limited to disease being treated; patient age, weight, sex, health; other medications and treatments being administered to the patient; and the like. A preferred dose is in the range of about 0.25−2.0×10⁶ cell equivalents. Other preferred dose ranges are 0.1−10.0×10⁶ cell equivalents based on in vitro as well as in vivo experiments. It has been found previously (see PCT/U.S. 2008/00396 and Banas, R. A., et al, (Human Immunology (2008) 69, 321-328, each incorporated by reference herein) that relatively small amounts of EHP cells can suppress inflammatory and immune responses, so it is expected that small amounts of membrane will also be effective.

EHP cell plasma membranes, including AMP cell plasma membranes, each alone or in combination, can be administered by injection into a target site of a subject, preferably via a delivery device, such as a tube, e.g., catheter. In a preferred embodiment, the tube additionally contains a needle, e.g., a syringe, through which the compositions can be introduced into the subject at a desired location. Specific, non-limiting examples of administering the compositions to subjects may also include administration by subcutaneous injection, intramuscular injection, or intravenous injection. If administration is intravenous, an injectable liquid suspension can be prepared and administered by a continuous drip or as a bolus.

The compositions of the invention may also be inserted into a delivery device, e.g., a syringe, in different forms. For example, the compositions can be suspended in a solution contained in such a delivery device. As used herein, the term “solution” includes a pharmaceutically acceptable carrier or diluent. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating EHP cell plasma membranes, including AMP cell plasma membranes, each alone or in combination, in a pharmaceutically acceptable carrier or diluent and, as required, other ingredients enumerated above, followed by filter sterilization.

Skilled artisans will also recognize that any and all of the standard methods and modalities for tissue, organ and cell-based transplantation therapies currently in clinical practice and clinical development are suitable for practicing the methods of the invention and using the novel compositions.

The invention is further illustrated by the following examples, which should not be construed as further limiting as the scope of the invention can only be determined by the claims.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Mixed Lymphocyte Reaction (MLR)

Normal peripheral blood mononuclear cells vs. HLA-DR (Class II) mismatched AMP cell plasma membrane.

HLA-G is a non-classical major histocompatability complex (MHC) class I molecule that has a tissue-restricted distribution. It has been shown to provide materno-fetal tolerance and is expressed on cytotrophoblasts at the fetal-maternal interface. HLA-G is also expressed by certain cancer lineages, where it may have a role in providing an escape mechanism to the immunosurveillance of the host organism. It has been shown (CMLS 55 1999 327-333) that HLA-G can inhibit MHC-restricted (T-cell) and unrestricted (NK cell) responses through Killing Inhibitory Receptors (KIR) expressed on the T-cell and NK cells. This includes lytic as well as proliferative responses. Therefore, HLA-G has immunomodulatory properties of the cells expressing it and of the environment in which these cells are located.

Applicants have found that AMP cells variably express the HLA-G cell surface marker upon isolation from the amnion (>60%, n=20); however, the expression of this marker decreases as the cells are cultured over time. When pro-inflammatory cytokines (IFNγ (100 IU/mL) alone or with TNFα (10 ng/mL) and IL1β (10 ng/mL)) are added to the culture, HLA-G expression is up-regulated. In addition, the MHC Class II markers (i.e. DR, DP, DQ) are not expressed by AMP cells either at isolation or after culture with or without pro-inflammatory cytokines Applicants have also found that AMP cells do not express the co-stimulatory molecules CD80 and CD86, which are pivotal in signaling a normal immune response and that AMP cells are not capable of generating an immune response at the helper T-cell level (for details see PCT/U.S. 2008/00396 and Banas, R. A., et al, (Human Immunology (2008) 69, 321-328, each incorporated by reference herein).

AMP cell plasma membranes are tested in the same standard MLR reactions as those described in PCT/U.S. 2008/00396 and Banas, R. A., et al, (Human Immunology (2008) 69, 321-328, to evaluate whether they, like the AMP cells from which they are derived, are incapable of generating an immune response at the helper T-cell level.

### Example 2

### Normal Mononuclear Cell MLR Plus the Addition of HLA-DR (Class II) Mismatched AMP Cell Membranes.

Applicants have previously demonstrated that AMP cells have an immunomodulatory capabilities (for details see PCT/U.S. 2008/00396 and Banas, R. A., et al, (Human Immunology (2008) 69, 321-328, each incorporated by reference herein). AMP cell plasma membranes are tested in the same standard MLR reactions as those described in PCT/U.S. 2008/00396 and Banas, R. A., et al, (Human Immunology (2008) 69, 321-328), to evaluate whether they, like the AMP cells from which they are derived, possess immunomodulatory capabilities.

### Example 3

### Effects of Serially Diluted AMP Cell Plasma Membranes on Allo-Antigen MLR.

Applicants have previously demonstrated that serially diluted AMP cells on allo-antigen MLR show significant inhibition of the normal MLR. The MLR was inhibited by up to 89% at the highest AMP cell dilution. A titration effect was observed, with AMP cell inhibition remaining over 20% even at a 1:64 dilution (for details see PCT/U.S. 2008/00396 and Banas, R. A., et al, (Human Immunology (2008) 69, 321-328, each incorporated by reference herein). Serially diluted AMP cell plasma membranes are tested in the same standard MLR reactions as those described in PCT/U.S. 2008/00396 and Banas, R. A., et al, (Human Immunology (2008) 69, 321-328), to evaluate whether they, like the AMP cells from which they are derived, demonstrated a titratable inhibitory effect.

The present invention may be embodied in other specific forms without departing from the spirit or essential attributes thereof Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

We claim,:

1. A composition comprising substantially purified extraembryonic (EE) cell plasma membranes.

2. The composition of claim 1 which is a pharmaceutical composition.

3. A kit comprising the pharmaceutical composition of claim 2.

4. The composition of claim 1 wherein the EE cell plasma membranes are selected from the group consisting of extraembryonic HLA-G positive (EHP) cell plasma membranes and Amnion-derived Multipotent Progenitor (AMP) cell plasma membranes.

5. The composition of claim 4 which is a pharmaceutical composition.

6. A kit comprising the pharmaceutical composition of claim 5.

7. A method of making a composition comprising substantially purified EE cell plasma membranes comprising:
a) isolating EE cells from extraembryonic tissue;
b) treating the EE cells such that their plasma membranes are separated from the other components of the EE cells; and
c) recovering the separated plasma membranes.

8. The method of claim 7 wherein the EE cells are selected from the group consisting of EHP cells and AMP cells.

9. A method of down-regulating an immune response or an inflammatory response in an subject in need thereof, such method comprising administering to the subject an effective amount of a composition selected from the group consisting of EE cell plasma membranes, EHP cell plasma membranes and AMP cell plasma membranes.

10. The method of claim 9 wherein the immune response is an autoimmune response which is selected from the group consisting of Type I diabetes, multiple sclerosis, systemic lupus erythematosus, Grave's disease, autoimmune hemolytic anemia, bullous pemphigoid, Hashimoto's thyroiditis, myasthenia gravis, pemphigus, and pernicious anemia.

11. The method of claim 9 wherein the immune response is an allogeneic immune response which is selected from the group consisting of graft versus host disease and host versus graft disease.

12. The method of claim 9 wherein the inflammatory response is selected from the group consisting of an inflammatory disease of the integument, an inflammatory bowel disease and a rheumatic disease.

13. The method of claim 9 wherein the composition is co-administered with one or more active agents, wherein the active agent is selected from the group consisting of a corticosteroid, a cyclosporine, a tacrolimus, a sirolimus, a methotrexate, a azathiopine, a mercatopurine, a cytotoxic antibiotic, a polyclonal antibody, a monoclonal antibody, an interferon, an opioid, a TNF binding protein, a mycophenolate, and FTY720.

14. The method of claim 13 wherein the monoclonal antibody is selected from the group consisting of an anti-T-cell receptor (CD23) and an anti-IL2 receptor (CD25) antibody.
